SEQUENCE LISTING (15 sequences)

SEQ ID NO: 1 - protein, 16 aa, artificial sequence
  Synthetic Peptide
CGRGRGRGRGRGRGRG

SEQ ID NO: 2 - DNA, 24 bp, artificial sequence
  primer
ggagctcatggcgcagagctggtt

SEQ ID NO: 3 - DNA, 24 bp, artificial sequence
  primer
gcaagctttcagaagctgaacgca

SEQ ID NO: 4 - DNA, 32 bp, artificial sequence
  primer
gagaattcatgctggaactggacggacgcacg

SEQ ID NO: 5 - DNA, 32 bp, artificial sequence
  primer
ggaagctttcaccccctcacgcagtgagcaac

SEQ ID NO: 6 - protein, 941 aa, artificial sequence
  Jumonji Fusion Protein
MKIEEGKLVIWINGDKGYNGLAEVGKKFEKDTGIKVTVEHPDKLEEKFPQVAATGDGPDIIFWAHDRFGGYAQSGLLAEITPDKAFQDKLYPFTWDAVRYNGKLIAYPIAVEALSLIYNKDLLPNPPKTWEEIPALDKELKAKGKSALMFNLQEPYFTWPLIAADGGYAFKYENGKYDIKDVGVDNAGAKAGLTFLVDLIKNKHMNADTDYSIAEAAFNKGETAMTINGPWAWSNIDTSKVNYGVTVLPTFKGQPSKPFVGVLSAGINAASPNKELAKEFLENYLLTDEGLEAVNKDKPLGAVALKSYEEELAKDPRIAATMENAQKGEIMPNIPQMSAFWYAVRTAVINAASGRQTVDEALKDAQTNSSSNNNNNNNNNNLGIEGRISEFMAQSWFKRHPLGIYPRGNAKNLDELVSPRFGILSTLQFASKGRAGKCCILGSQPAEEAFVDVMLHLLSFVAVDDMCRLSAVCTGWYCFIHASDAFKQAHGLLSPTYTCFEGSWKETAIRRFIKLRSKQRLNTAKRMRLEGGTPGVGETVNLSHRPVMVKRAFYNDQLFQAWMCTILPCHYHLRQSSIAGTATANRQPSVLSARGRYRSPLKEVPRCSGLSVDEFRTRFEETNLPVIIADVATEWPIYKILQEKFENLAVMQKKLFRPGTRPDVPMCCEHTTMSVADYVRYARDQTDERPIYLFDSEFGTFMDVESLYTVPEYFSRDDFFKVLGGARPKYRWIIAGPRRGGSSFHVDPNYTSAWNANLTGLKRWILLPPGHTPAGVFPSEDMSEVVTPVSLTEWLLNHYDATVEKWRDVAYECVCGPGDIMFIPCGWWHFVINLEDSVAITQNYVSKCNLSSVLKFLSVMKSSISGIDEDVDNCDTSRMVETRRANFAEEFAAAMHLSYPELMQTVAGEAKREAEERHEKKKMRAALPLLDVGSNGFAFSF

SEQ ID NO: 7 - protein, 841 aa, artificial sequence
  Jumonji Fusion Protein
MKIEEGKLVIWINGDKGYNGLAEVGKKFEKDTGIKVTVEHPDKLEEKFPQVAATGDGPDIIFWAHDRFGGYAQSGLLAEITPDKAFQDKLYPFTWDAVRYNGKLIAYPIAVEALSLIYNKDLLPNPPKTWEEIPALDKELKAKGKSALMFNLQEPYFTWPLIAADGGYAFKYENGKYDIKDVGVDNAGAKAGLTFLVDLIKNKHMNADTDYSIAEAAFNKGETAMTINGPWAWSNIDTSKVNYGVTVLPTFKGQPSKPFVGVLSAGINAASPNKELAKEFLENYLLTDEGLEAVNKDKPLGAVALKSYEEELAKDPRIAATMENAQKGEIMPNIPQMSAFWYAVRTAVINAASGRQTVDEALKDAQTNSSSNNNNNNNNNNLGIEGRISEFMLELDGRTLTFEQFRECCLKPNLPAIIRHAAVDSTTNSVGGSGPSPYFSPLGDMQSHLSPIGVVNLFGGDHVVPATESPATVASDSLKDGISEGHMKCSKLRLFEVIERWRGSPTLVYVKDWHMQSDLEAVSSCVGAPDLRGVSSVVASDAEEGNAAVCNGRRAVVHGGNLYCVPCYLGPDWMDEFCRFSQHGDSKYRYFGEEESDYRFAYIGPPRSWTPLHFDVFGTYSWSLNVCGEKLWFFPTPEGNQTLLRGGLHGVALAPDIRTTAGAELWTVTQYPGDLVFVPSCYLHQVHNVKGSCFTLPQTRETANVAATSCEGIESVSTPNESVVDLVISINHNWCNEWCVERMVDAFCRDANRLWMLLGDEVRLTLFGDDVGAWHDHVENLLMGGTNWNFGCIRSFLVYRLQVLRSSGPTTAEGDNVRRLIELCLGKVNETEMRVAHCVRG

SEQ ID NO: 8 - protein, 550 aa, Trypanosoma brucei
MAQSWFKRHPLGIYPRGNAKNLDELVSPRFGILSTLQFASKGRAGKCCILGSQPAEEAFVDVMLHLLSFVAVDDMCRLSAVCTGWYCFIHASDAFKQAHGLLSPTYTCFEGSWKETAIRRFIKLRSKQRLNTAKRMRLEGGTPGVGETVNLSHRPVMVKRAFYNDQLFQAWMCTILPCHYHLRQSSIAGTATANRQPSVLSARGRYRSPLKEVPRCSGLSVDEFRTRFEETNLPVIIADVATEWPIYKILQEKFENLAVMQKKLFRPGTRPDVPMCCEHTTMSVADYVRYARDQTDERPIYLFDSEFGTFMDVESLYTVPEYFSRDDFFKVLGGARPKYRWIIAGPRRGGSSFHVDPNYTSAWNANLTGLKRWILLPPGHTPAGVFPSEDMSEVVTPVSLTEWLLNHYDATVEKWRDVAYECVCGPGDIMFIPCGWWHFVINLEDSVAITQNYVSKCNLSSVLKFLSVMKSSISGIDEDVDNCDTSRMVETRRANFAEEFAAAMHLSYPELMQTVAGEAKREAEERHEKKKMRAALPLLDVGSNGFAFSF

SEQ ID NO: 9 - protein, 450 aa, Trypanosoma brucei
MLELDGRTLTFEQFRECCLKPNLPAIIRHAAVDSTTNSVGGSGPSPYFSPLGDMQSHLSPIGVVNLFGGDHVVPATESPATVASDSLKDGISEGHMKCSKLRLFEVIERWRGSPTLVYVKDWHMQSDLEAVSSCVGAPDLRGVSSVVASDAEEGNAAVCNGRRAVVHGGNLYCVPCYLGPDWMDEFCRFSQHGDSKYRYFGEEESDYRFAYIGPPRSWTPLHFDVFGTYSWSLNVCGEKLWFFPTPEGNQTLLRGGLHGVALAPDIRTTAGAELWTVTQYPGDLVFVPSCYLHQVHNVKGSCFTLPQTRETANVAATSCEGIESVSTPNESVVDLVISINHNWCNEWCVERMVDAFCRDANRLWMLLGDEVRLTLFGDDVGAWHDHVENLLMGGTNWNFGCIRSFLVYRLQVLRSSGPTTAEGDNVRRLIELCLGKVNETEMRVAHCVRG

SEQ ID NO: 10 - protein, 941 aa, artificial sequence
  Fusion Protein
MKIEEGKLVIWINGDKGYNGLAEVGKKFEKDTGIKVTVEHPDKLEEKFPQVAATGDGPDIIFWAHDRFGGYAQSGLLAEITPDKAFQDKLYPFTWDAVRYNGKLIAYPIAVEALSLIYNKDLLPNPPKTWEEIPALDKELKAKGKSALMFNLQEPYFTWPLIAADGGYAFKYENGKYDIKDVGVDNAGAKAGLTFLVDLIKNKHMNADTDYSIAEAAFNKGETAMTINGPWAWSNIDTSKVNYGVTVLPTFKGQPSKPFVGVLSAGINAASPNKELAKEFLENYLLTDEGLEAVNKDKPLGAVALKSYEEELAKDPRIAATMENAQKGEIMPNIPQMSAFWYAVRTAVINAASGRQTVDEALKDAQTNSSSNNNNNNNNNNLGIEGRISEFMAQSWFKRHPLGIYPRGNAKNLDELVSPRFGILSTLQFASKGRAGKCCILGSQPAEEAFVDVMLHLLSFVAVDDMCRLSAVCTGWYCFIHASDAFKQAHGLLSPTYTCFEGSWKETAIRRFIKLRSKQRLNTAKRMRLEGGTPGVGETVNLSHRPVMVKRAFYNDQLFQAWMCTILPCHYHLRQSSIAGTATANRQPSVLSARGRYRSPLKEVPRCSGLSVDEFRTRFEETNLPVIIADVATEWPIYKILQEKFENLAVMQKKLFRPGTRPDVPMCCEHTTMSVADYVRYARDQTDERPIYLFDSEFGTFMDVESLYTVPEYFSRDDFFKVLGGARPKYRWIIAGPRRGGSSFAVDPNYTSAWNANLTGLKRWILLPPGHTPAGVFPSEDMSEVVTPVSLTEWLLNHYDATVEKWRDVAYECVCGPGDIMFIPCGWWHFVINLEDSVAITQNYVSKCNLSSVLKFLSVMKSSISGIDEDVDNCDTSRMVETRRANFAEEFAAAMHLSYPELMQTVAGEAKREAEERHEKKKMRAALPLLDVGSNGFAFSF

SEQ ID NO: 11 - protein, 841 aa, artificial sequence
  Fusion Protein
MKIEEGKLVIWINGDKGYNGLAEVGKKFEKDTGIKVTVEHPDKLEEKFPQVAATGDGPDIIFWAHDRFGGYAQSGLLAEITPDKAFQDKLYPFTWDAVRYNGKLIAYPIAVEALSLIYNKDLLPNPPKTWEEIPALDKELKAKGKSALMFNLQEPYFTWPLIAADGGYAFKYENGKYDIKDVGVDNAGAKAGLTFLVDLIKNKHMNADTDYSIAEAAFNKGETAMTINGPWAWSNIDTSKVNYGVTVLPTFKGQPSKPFVGVLSAGINAASPNKELAKEFLENYLLTDEGLEAVNKDKPLGAVALKSYEEELAKDPRIAATMENAQKGEIMPNIPQMSAFWYAVRTAVINAASGRQTVDEALKDAQTNSSSNNNNNNNNNNLGIEGRISEFMLELDGRTLTFEQFRECCLKPNLPAIIRHAAVDSTTNSVGGSGPSPYFSPLGDMQSHLSPIGVVNLFGGDHVVPATESPATVASDSLKDGISEGHMKCSKLRLFEVIERWRGSPTLVYVKDWHMQSDLEAVSSCVGAPDLRGVSSVVASDAEEGNAAVCNGRRAVVHGGNLYCVPCYLGPDWMDEFCRFSQHGDSKYRYFGEEESDYRFAYIGPPRSWTPLAFDVFGTYSWSLNVCGEKLWFFPTPEGNQTLLRGGLHGVALAPDIRTTAGAELWTVTQYPGDLVFVPSCYLHQVHNVKGSCFTLPQTRETANVAATSCEGIESVSTPNESVVDLVISINHNWCNEWCVERMVDAFCRDANRLWMLLGDEVRLTLFGDDVGAWHDHVENLLMGGTNWNFGCIRSFLVYRLQVLRSSGPTTAEGDNVRRLIELCLGKVNETEMRVAHCVRG

SEQ ID NO: 12 - protein, 550 aa, artificial sequence
  Engineered Point Mutation
MAQSWFKRHPLGIYPRGNAKNLDELVSPRFGILSTLQFASKGRAGKCCILGSQPAEEAFVDVMLHLLSFVAVDDMCRLSAVCTGWYCFIHASDAFKQAHGLLSPTYTCFEGSWKETAIRRFIKLRSKQRLNTAKRMRLEGGTPGVGETVNLSHRPVMVKRAFYNDQLFQAWMCTILPCHYHLRQSSIAGTATANRQPSVLSARGRYRSPLKEVPRCSGLSVDEFRTRFEETNLPVIIADVATEWPIYKILQEKFENLAVMQKKLFRPGTRPDVPMCCEHTTMSVADYVRYARDQTDERPIYLFDSEFGTFMDVESLYTVPEYFSRDDFFKVLGGARPKYRWIIAGPRRGGSSFAVDPNYTSAWNANLTGLKRWILLPPGHTPAGVFPSEDMSEVVTPVSLTEWLLNHYDATVEKWRDVAYECVCGPGDIMFIPCGWWHFVINLEDSVAITQNYVSKCNLSSVLKFLSVMKSSISGIDEDVDNCDTSRMVETRRANFAEEFAAAMHLSYPELMQTVAGEAKREAEERHEKKKMRAALPLLDVGSNGFAFSF

SEQ ID NO: 13 - protein, 450 aa, artificial sequence
  Engineered Point Mutation
MLELDGRTLTFEQFRECCLKPNLPAIIRHAAVDSTTNSVGGSGPSPYFSPLGDMQSHLSPIGVVNLFGGDHVVPATESPATVASDSLKDGISEGHMKCSKLRLFEVIERWRGSPTLVYVKDWHMQSDLEAVSSCVGAPDLRGVSSVVASDAEEGNAAVCNGRRAVVHGGNLYCVPCYLGPDWMDEFCRFSQHGDSKYRYFGEEESDYRFAYIGPPRSWTPLAFDVFGTYSWSLNVCGEKLWFFPTPEGNQTLLRGGLHGVALAPDIRTTAGAELWTVTQYPGDLVFVPSCYLHQVHNVKGSCFTLPQTRETANVAATSCEGIESVSTPNESVVDLVISINHNWCNEWCVERMVDAFCRDANRLWMLLGDEVRLTLFGDDVGAWHDHVENLLMGGTNWNFGCIRSFLVYRLQVLRSSGPTTAEGDNVRRLIELCLGKVNETEMRVAHCVRG

SEQ ID NO: 14 - protein, 15 aa, artificial sequence
  Synthetic Peptide
GRGRGRGRGRGRGRG

SEQ ID NO: 15 - protein, 7 aa, artificial sequence
  Synthetic Peptide
GRGRGRG

(19) United States
(12) Patent Application Publication
(10) Pub. No.: US 2017/0218430 A1
(43) Pub. Date: Aug. 3, 2017

(54) DETECTION OF PROTEIN ARGININE DEMETHYLASE ACTIVITY

(71) Applicant: The Research Foundation for The State University of New York, Amherst, NY (US)

(72) Inventors: John M. Aletta, Williamsville, NY (US); John C. Fisk, N. Tonawanda, NY (US); Laurie K. Read, Orchard Park, NY (US)

(21) Appl. No.: 15/501,041

(22) PCT Filed: Jul. 31, 2015
(86) PCT No.: PCT/US2015/043154

§ 371 Date: Feb. 1, 2017

Related U.S. Application Data
(60) Provisional application No. 62/032,209, filed on Aug. 1, 2014.

(51) Int. Cl. C12Q 1/26; G01N 33/68; C07K 16/44; C12N 9/02

(52) U.S. Cl. CPC C12Q 1/26; C12N 9/0071; G01N 33/6812; C12Y 114/00; C07K 16/44; G01N 2333/90245; G01N 2440/12; G01N 2500/20

(57) ABSTRACT

Provided are methods and compositions for determining methylarginine demethylase activity in test samples. The methods and compositions comprise a peptide substrate containing methylated arginine that can act as a substrate for the demethylation activity, a positive control that has methylarginine demethylation activity and a variant of the positive control that does not have methylarginine demethylation activity and that can act as a negative control.

### CROSS-REFERENCE TO RELATED APPLICATIONS

This application claims priority to U.S. provisional patent application No. 62/032,209 filed on Aug. 1, 2014, the disclosure of which is incorporated herein by reference.

### STATEMENT REGARDING FEDERALLY SPONSORED RESEARCH

This invention was made with government support under grant number R01A160260 from the National Institutes of Health. The government has certain rights in the invention.

### FIELD OF THE DISCLOSURE

This disclosure relates generally to the field of protein arginine methylation and in particular relates to detecting protein arginine demethylation activity.

### BACKGROUND OF THE DISCLOSURE

Arginine methylation of proteins is important for biomedical science and human health because the consequences of this post-translational modification have functional implications for physiological responses including gene activation/deactivation, protein translocation and cell signaling. Protein arginine demethylation represents a unique new drug target for many human diseases including cancer. Demethylation is significant because of the potential usefulness of the enzyme(s) in the development of first-in-class pharmacological interventions capable of interfering with specific disease processes.

The determination of arginine demethylation activity in cells and organs is fundamentally important for an accurate understanding of the steady state of protein arginine methylation status. Several enzymatic assays to determine protein arginine methyltransferase activity are available to measure the addition (“on rate”) of methyl-groups to arginine residues in proteins, but the removal (“off rate”) of methyl-groups is difficult to determine with regard to methylarginine proteins.

### BRIEF DESCRIPTION OF THE FIGURES

FIG. 1. Representation of binding of methylarginine-specific antibody. A series dilution (3 ng to 0.1 ng) of methylarginine peptide (SEQ ID NO:1) was prepared in 10 mM Tris pH 8 and applied to PVDF membrane held in a slot blot apparatus (left column FIG. 1). Six reaction tubes were prepared containing 50 mM HEPES pH 8, 1 mM oxoglutarate, 0.5 mM ascorbate and 20 μM ammonium iron (II) sulfate hexahydrate. TbJmJ fusion proteins were expressed in bacterial cells and purified on an amylose agarose resin column. TbJmJ fusion proteins were diluted to 25 ng/μl. TbJmJ2 fusion protein was added to the reaction tubes (2, 4 or 6 μl)±1 ng methylarginine peptide. After a 20 hour incubation period at 22.5°, the contents of the reaction tubes were added to the PVDF membrane held in the slot blot apparatus. This figure illustrates the binding of methylarginine-specific antibody to methylarginine peptides.

FIG. 2. Representation of binding of the antibody specific for the non-methylated form of the peptide. An experiment similar to that described in FIG. 1 was carried out to detect the presence of de-methylated peptide in the reactions by using an antibody specific for the non-methylated form of the peptide. Methylarginine (GmRG) and arginine (GRG) peptide standards from 100 pg to 1 ng were prepared in 10 mM Tris pH 8 and reaction tubes containing TbJmJ1 fusion protein and TbJmJ2 fusion protein (100 to 500 pg)±1 ng methylarginine peptide in HEPES buffer pH 8 with 1 mM oxoglutarate, 0.5 mM ascorbate and 20 μM ammonium iron (II) sulfate hexahydrate were incubated for 6 hours at 30° C. This figure illustrates the binding of the antibody specific for the non-methylated form of the peptide.

### DETAILED DESCRIPTION OF THE DISCLOSURE

The present disclosure provides methods and compositions for detection of demethylation activity in test samples. In one embodiment, the method uses an antibody that specifically binds to arginine peptides or proteins only when the arginines are in the non-methylated state. When the peptide or protein is arginine methylated, the antibody does not specifically bind the peptide or protein. Alternatively, antibodies that specifically bind to arginine peptides when the arginines are in the methylated state can also be used.

The disclosure provides a method for detecting arginine demethylation. For example, the method can be used for detecting if a biological sample contains demethylation activity. An arginine peptide in the methylated state is used as a substrate for demethylation to determine the activity of demethylases. In one embodiment, a positive control is used in the form of a demethylating enzyme protein. The demethylating protein may be a recombinant enzyme cloned from T. brucei that is capable of demethylating in vitro a methylarginine peptide. In one embodiment, the recombinant enzyme may be in the form of Jumonji proteins produced from 2 genes cloned from the parasite, Trypanosoma brucei. In addition, two other protein products expressed from the cDNA of T. brucei have been produced to confirm the enzymatic nature of the T. brucei Jumonji proteins (TbJmJs). We found that a single mutation in the active site of the TbJmJs, produces inactive enzyme.

In one aspect, the present disclosure provides a method for determining if a test sample contains methylarginine demethylating activity comprising contacting the sample with a suitable substrate for arginine demethylation and monitoring the methylation (or demethylation) status. An example of a suitable substrate for detecting demethylation activity is an arginine methylated peptide having a GRG motif In one embodiment, the GRG peptide is from 5 to 30 amino acids (and all integers therebetween) in length and contains from 2 to 15 (and all integers therebetween) methylarginines. The methylarginines may be di-methylated or may be mono-methylated. After contacting the test sample with a suitable substrate, the effect on the methylation status can be determined, such as, for example, by determining if the substrate peptide is demethylated in the presence of the test sample using an antibody specific for demethylated arginine or specific for methylated arginine. In one embodiment, the antibody specific for demethylated arginine (anti-RG) is as described in P. Duan et al. (J. Immunological Methods, 320:132-142, 2007, disclosure incorporated herein by reference). It is commercially available (such as from CH3 BioSystems, NY). In one embodiment, the antibody specific for methylated arginine (anti-mRG) is as described in U.S. Pat. No. 6,699,672, the disclosure of which is incorporated herein by reference. This antibody is also available commercially (such as from CH3 BioSystems, NY). When an antibody herein is indicated to be specific, such as for example, specific for methylated arginine or demethylated arginine, that means that the antibody will not bind (other than non-specific background binding) to the methylated or the demethylated form as relevant. For example, if an antibody is specific for demethylated arginine, it will not bind (other than non-specific binding) to peptides/proteins containing arginine in methylated state, and if an antibody is specific for methylated arginine, it will not bind (other than non-specific background binding) to peptides or protein having arginine in a demethylated state. Based on the specific binding by either the anti-RG or anti-mRG antibody, the sample can be identified as having or lacking arginine demethylating activity. For example, an increase in the binding of anti-RG antibody or a decrease in the binding of anti-mRG is indicative of demethylation.

In one embodiment, the antibody that specifically binds to demethylated arginine peptides may be one of the following: It may be an isolated antibody which specifically binds to a peptide or protein comprising an epitope of at least 5 amino acids, the epitope having one or more GRG motifs wherein none of the arginine residues are methylated, but does not specifically bind to the peptide or protein when the arginine(s) is (are) methylated. In one embodiment, it may be an isolated antibody generated by immunization of an animal with a synthetic arginine peptide, which antibody specifically binds to a peptide or protein only when the peptide or protein is not arginine methylated. In one embodiment, the antibody is such that the differential binding of said antibody to Sequence #1 and to non-methylated degenerate congeners of Sequence #1 distinguishes reduced methylation from full methylation.

The antibodies may be monoclonal, polyclonal, or engineered including humanized antibodies or single domain antibodies (e.g., nanobodies). The antibodies may be generated in any species. For example, the antibodies may be generated in any mammal. Monoclonal antibodies may be prepared by techniques well known in the art. Further, chimeric or humanized antibodies may be prepared by techniques known in the art.

While any peptide with at least 5 amino acids and having a GRG motif and further wherein the arginine is methylated can be used, in one embodiment, the peptide is a synthetic peptide constructed of tandem repeats of glycine-dimethylarginine-glycine. In one embodiment, the peptide has from 5 to 30 amino acids. In one embodiment, the 5 to 30 amino acid peptide has a continuous sequence of alternating glycines and arginines or arginines and glycines. The methyl groups of the dimethylarginine may be oriented in either the symmetric or asymmetric configuration about the terminal nitrogen atoms of the arginine aliphatic side chain. In one embodiment, the peptide is: ^{H}Cys Gly DMA Gly DMA Gly DMA Gly DMA Gly DMA Gly DMA Gly DMA Gly^{NH2} in which ^{H}Cys=cysteine, Gly=glycine, Gly^{NH2}=glycinamide and DMA=dimethylarginine in either the asymmetric or symmetric configurations. (Sequence #1; SEQ ID NO:1). In one embodiment, the peptide does not have cysteine and therefore has the sequence GRGRGRGRGRGRGRG (SEQ ID NO:14). In one embodiment, the peptide is a single GRG or multiple repeats of GRG—with or without cysteines. The peptide may have 2, 3, 4, 5, 6, 7, 8, 9, 10, 11, 12, 13 or 14 repeats. For example, 3 GRG repeats is GRGRGRG (SEQ ID NO:15), and 7 GRG repeats is GRGRGRGRGRGRGRG (SEQ ID NO:14). The arginines in the peptides may be dimethylated or mono-methylated.

In one embodiment, the assay also comprises a positive control for arginine demethylation activity. In one embodiment, the positive control is a protein that exhibits arginine demethylation activity. For example, a positive enzyme control that exhibits arginine demethylation activity is Jumonji fusion protein or Jumonji proteins. A test sample that is identified as having methylarginine demethylase activity may also serve as a positive control. The assay may comprise a negative control which may be a reaction set in which the test sample is absent. In one embodiment, the assay comprises a negative control which is in the form of a protein that does not exhibit methylarginine demethylase activity. For example, the negative control may be a variant of the positive control enzyme protein, wherein the variant does not have methylarginine demethylase activity. A test sample that is identified as not having demethylase activity may also serve as a negative control.

The test sample in which demethylation activity is to be detected may be any test sample. For example, it may be biological fluids, including but not limited to saliva, blood, secretions and cerebrospinal fluid, cell culture material, tissue samples including biopsy, autopsy and necropsy samples. In the case of cellular and tissue-based samples, homogenization and/or fractionation may be carried out to remove solid particulate materials. Samples can be freshly prepared from the host organism or cultured material, or from previously prepared sources and stored refrigerated or frozen. To carry out the assay to detect demethylation, the test sample is incubated with a substrate for the demethylation activity.

For example, the test sample may be incubated with a peptide or protein that contains GRG motif. The incubation may be carried out from periods of a few minutes to a day or longer. For example, the incubation may be carried out for 1-24 hours and all values to tenth decimal place there between. The temperature can be adjusted to suit the assay needs. In one embodiment, it can conveniently be from about room temperature (generally around 22° C.) to physiological temperature (generally around 37° C.) or from 22° C. to 37° C. and all temperatures to the tenth decimal place there between. Lower temperatures (i.e. from 4 and 10° C., or from 10° C. to room temperature) may also be employed for incubations of longer durations. Following incubation, an antibody (anti-RG or anti-mRG) can be used to determine if the peptide/protein has been demethylated. Various techniques are known in the art to detect specific antibody-antigen binding that include various types of immunoassays including ELISA and lanthanide-based Time Resolved Fluorescence Assays. In the various immunoassays, non-specific binding is minimized by the use of non-specific blockers such as bovine serum albumin, egg whites and the like.

The various incubations between the test samples and the substrate, and for detection of binding of anti-RG or anti-mRG may be carried out in standard buffers. For example, the incubations may be carried out in phosphate buffered saline or any of Good's Buffers (e.g. MES, PIPES, MOPS, HEPES and the like) or any other buffer suitable for enzyme substrate or antibody binding reactions. The optimum time and temperature of incubation may be determined empirically by those skilled in the art without undue experimentation.

In one embodiment, the assay is as follows: 1. Incubate methylarginine peptide (Sequence #1) in solution containing suspected arginine demethylase (biological fluid such as homogenates or extracts from tissues or cells) for time periods from 1 to 24 hours at room temperature or 37° C.; 2. Collect each sample of reactants in buffer. Transfer to SDS-PAGE for western blot procedure using PolyVinyleneDiFluoride (PVDF) membrane or apply directly to PVDF membrane fitted on a slot blotter under vacuum; 3. Block PVDF membrane with solution of 1% bovine serum albumin in phosphate buffered saline over night at 4° C.; 4. Incubate PVDF membrane with antibody raised against non-methylated glycine-arginine-rich peptide equivalent to Sequence #1, but with non-methylated arginine substituted for every DMA residue; and 5. Detect antibody binding to non-methylated peptide (produced by demethylation) using standard chromagen, chemi-luminescence, fluorescent or other detection protocol.

In one aspect, this disclosure provides a method for determining if a test sample contains methylarginine demethylation activity. The method comprises: a) contacting the test sample with a peptide substrate for methylarginine demethylase activity comprising a GRG motif, wherein the R is methylated; b) separately contacting the peptide substrate with a positive control for methylarginine demethylase activity; c) optionally, separately contacting the peptide with a negative control for methylarginine demethylase activity; and d) using an antibody that is specific for methylated arginine in a GRG motif or specific for non-methylated arginine in a GRG motif, determining if there is a decrease in the methylation level of the substrate peptide by the test and the positive control. A decrease in the level of methylation can be detected as decrease in the level of anti-mRG binding or an increase in the level of anti-RG binding. A decrease in the methylation status of the peptide substrate by the test sample (over time or as compared to a control) identifies the presence of methylarginine demethylation activity in the test sample, and a lack of decrease in the methylation status of the peptide substrate alone or combined with a decrease of methylation status by the positive enzyme control (e.g., proteins comprising SEQ ID NOs:6-9, or positive test samples) identifies the absence of methylarginine demethylation activity in the test sample. When a negative control (e.g., proteins comprising SEQ ID NOs: 10-13, or negative test samples) is used, a decrease in the methylation status of the substrate by the test sample combined with a lack of decrease in the methylation status of the substrate by the negative control identifies the presence of methylarginine demethylation activity. The peptide substrate may comprise or consist of the sequence of SEQ ID NO:1. In one embodiment, the peptide substrate does not have cys in SEQ ID NO:1.

In one aspect, this disclosure provides a method for determining if a test sample contains an agent that interferes with arginine demethylase activity by contacting arginine methylated peptide comprising a GRG motif (such as a peptide of SEQ ID NO:1, with or without cys) with a positive enzyme control for demethylation activity (such as one of the Jumonji fusion protein 1 or 2, or Jumonji proteins—SEQ ID NOs: 8 or 9) in the presence or absence of the test sample, and using an antibody which can specifically distinguish between arginine methylated state and arginine demethylated state of peptides or proteins to identify if demethylation of the peptide is affected in the presence of the test sample. The method of this embodiment can be used for screening for test agents that interfere with arginine demethylase activity. In one embodiment, a negative control—such as Jumonji fusion protein 3 or 4, or proteins comprising SEQ ID NOs: 12 or 13—may also be included.

In one aspect, this disclosure provides a kit for determining the arginine demethylation status comprising one or more of: a) methylarginine peptides comprising a GRG motif, b) positive enzyme control for methylarginine demethylation activity; c) negative control for methylarginine demethylation activity; d) one or more antibodies capable of distinguishing between the methylated or demethylated arginine peptides or proteins; and e) instructions for the assay. In one embodiment, the peptide comprises or consists of SEQ ID NO:1. In one embodiment, the peptide comprises or consists of SEQ ID NO:1 without the terminal cys. In one embodiment, the positive enzyme control is one or more Jumonji proteins or Jumonji fusion protein (such as Jumonji protein 1 or 2). In one embodiment, the positive enzyme control is a protein comprising or consisting of the sequence of SEQ ID NO: 8 or SEQ ID NO:9. In one embodiment, the negative control is one or more Jumonji fusion protein (such as Jumonji protein 3 or 4). In one embodiment the negative control is a protein comprising or consisting of the sequence of SEQ ID NO:12 or SEQ ID NO:13. The kit may also contain buffers and other assay reagents. For example, kit ingredients may additionally include: 1. phosphate buffered saline or any of Good's Buffers (e.g. MES, PIPES, MOPS, HEPES) typically used for biochemical and biological research—2. Protease inhibitors such as phenylmethylsulfonyl fluoride or benzamidine among many others 3. Cofactors necessary for dioxygenase-type enzymes such as Fe(II), 2-oxoglutarate and ascorbate. The instructions for use may include one or more of the following: details on various steps of the assay process, guidance on the interpretation of results, storage guidance etc. for the antibody, enzymes and other ingredients and the like. Other instructions may include recommendations for reaction temperatures and incubation times, the range of useful protein concentrations for different types of biological samples committed to the assay providing for sufficient activity above a lower limit of detection and below an upper limit to obviate non-specific protein binding.

The following examples are provided to further illustrate the invention and they are not intended to be restrictive in any way.

### EXAMPLE 1

This example describes the production of Jumonji fusion proteins that have methylarginine demethylation activity. Recombinant construction of four plasmids based on two T. brucei genes enables production of four unique bacterially expressed fusion proteins for use in experimental and drug development settings. The two genes belong to the family of Jumonji proteins (TbJmJ1 and TbJmJ2) found in primordial eukaryotes and Homo sapiens. The recombinant proteins derived from the two genes have been engineered to include the N-terminal peptide tag, maltose-binding protein, that facilitates purification from lysates of bacterial preparations. Two of the recombinant fusion proteins faithfully contain the complete cDNAs of two separate T. brucei Jumonji genes. A third fusion protein contains a site-directed mutation of histidine 745 (of SEQ ID NO:6) to alanine at the active site of the TbJmJ1 (H745ATbJmJ1), which inactivates any catalytic demethylation activity associated with the protein. Similarly, the fourth fusion protein, an inactive form of TbJmJ2, is produced by mutation of histidine 613 (of SEQ ID NO:7) (H613ATbJmJ2). Fusion proteins 3 and 4 will serve as negative controls for the active enzymes present in the assay reactions. At high concentrations, fusion proteins 3 and 4 may serve as dominant negative regulators of demethylation by binding and sequestering substrates making the methylarginine sites unavailable to endogenous arginine demethylases. Also, when the DNA sequence which codes for the TbJmJ1 and TbJmJ2 proteins is inserted into a mammalian expression vector such as pcDNA3.1 or pcDNA3.2 or other expression vectors suitable for animal or plant cell expression, overexpression in eukaryotic cells is expected to inhibit endogenous arginine demethylase activities.

The fusion proteins described herein consist of bacterial expressed Trypanosoma brucei protein fused with a peptide tag that can be used for the isolation and purification of the protein. In the one embodiment that is described, the peptide tag is maltose-binding protein (MBP). Other embodiments of the invention may utilize glutathione-S-transferase (GST), histidine (HIS), 6X HIS, horseradish peroxidase (HRP), hemagglutinin (HA), Flag tag (DDK), or the peptide tag derived from the c-myc gene product (Myc).

Cloning and expression of recombinant proteins. The open reading frames of TbJmJ1, Tb927.11.2000* and TbJmJ2, Tb927.11.1760* were PCR amplified from oligo(dT)-primed cDNA extracted from procyclic form T. brucei (29-13) RNA using the primers:

JMJ1-5′-SacI
(SEQ ID NO: 2)
(5′-GGAGCTCATGGCGCAGAGCTGGTT-3′),
and
JMJ1-3′-HindIII
(SEQ ID NO: 3)
(5′-GCAAGCTTTCAGAAGCTGAACGCA-3′) for TbJMJ1
JMJ2-5′-EcoRI
(SEQ ID NO: 4)
(5′-GAGAATTCATGCTGGAACTGGACGGACGCACG-3′),
and
JMJ2-3′-HindIII
(SEQ ID NO: 5)
(5′-GGAAGCTTTCACCCCCTCACGCAGTGAGCAAC-3′) TbJMJ2.

The sequences of TbJmJ1 (XP_8282884.1 Mar. 26, 2012) and TbJmJ2 (XP_828265.1 Mar. 26, 2012) are publicly available. The protein sequence of the fusion proteins is provided below. N-terminal MBP is in each sequence with start site of the TbJmJ protein being indicated as underlined M and active histidine as underlined H.

The restriction enzyme cut sites are underlined above. The resultant product was cloned into pJET1.2 (Fermentas; ThermoFisher). Both genes were excised from their respective pJET vectors and ligated into the corresponding sites of pMAL-c2 (New England Biolabs). The resultant plasmids were then transformed into Rosetta strain Escherichia coli cells (Novagen) for expression. MBP-tagged JmJ1 and MBP-JmJ2 were purified using single step purification as follows. Bacterial cells were grown in LB culture broth with 0.2% glucose to an O.D. of 0.5 and induced for protein production overnight at 18 degrees C. using 0.1 mM IPTG. Cells were collected and lysed in PBS buffer containing 1 mM PMSF and 1 mM DTT using 3 rounds of sonication. The NaCl concentration was adjusted to 1M and MBP tagged proteins were bound to a 1 mL amylose agarose resin (New England Biolabs) for 2 hours at 4 degrees C. The flow through was collected and the resin washed with 100 times bed volume (100 mL) using PBS with 1M NaCl. Prior to elution, the resin was equilibrated in PBS with standard salt concentration. MBP-tagged proteins were then eluted with PBS containing 10 mM maltose and concentrations of eluted proteins determined using a standard curve against known concentrations of bovine serum albumin.

The term TbJmJ stands for Trypanosoma brucei Jumonji. *Trypanosoma brucei gene identifier can be found in Kinetoplastid Genome Resource database (tritrypdb.org)

The sequences of two Jumonji fusion proteins are provided below:

MBP-JmJ1 (Jumonji Fusion Protein 1)
(SEQ ID NO: 6)
MKIEEGKLVI WINGDKGYNG LAEVGKKFEK DTGIKVTVEH PDKLEEKFPQ 50
VAATGDGPDI IFWAHDRFGG YAQSGLLAEI TPDKAFQDKL YPFTWDAVRY 100
NGKLIAYPIA VEALSLIYNK DLLPNPPKTW EEIPALDKEL KAKGKSALMF 150
NLQEPYFTWP LIAADGGYAF KYENGKYDIK DVGVDNAGAK AGLTFLVDLI 200
KNKHMNADTD YSIAEAAFNK GETAMTINGP WAWSNIDTSK VNYGVTVLPT 250
FKGQPSKPFV GVLSAGINAA SPNKELAKEF LENYLLTDEG LEAVNKDKPL 300
GAVALKSYEE ELAKDPRIAA TMENAQKGEI MPNIPQMSAF WYAVRTAVIN 350
AASGRQTVDE ALKDAQTNSS SNNNNNNNNN NLGIEGRISE FMAQSWFKRH 400
PLGIYPRGNA KNLDELVSPR FGILSTLQFA SKGRAGKCCI LGSQPAEEAF 450
VDVMLHLLSF VAVDDMCRLS AVCTGWYCFI HASDAFKQAH GLLSPTYTCF 500
EGSWKETAIR RFIKLRSKQR LNTAKRMRLE GGTPGVGETV NLSHRPVMVK 550
RAFYNDQLFQ AWMCTILPCH YHLRQSSIAG TATANRQPSV LSARGRYRSP 600
LKEVPRCSGL SVDEFRTRFE ETNLPVIIAD VATEWPIYKI LQEKFENLAV 650
MQKKLFRPGT RPDVPMCCEH TTMSVADYVR YARDQTDERP IYLFDSEFGT 700
FMDVESLYTV PEYFSRDDFF KVLGGARPKY RWIIAGPRRG GSSFHVDPNY 750
TSAWNANLTG LKRWILLPPG HTPAGVFPSE DMSEVVTPVS LTEWLLNHYD 800
ATVEKWRDVA YECVCGPGDI MFIPCGWWHF VINLEDSVAI TQNYVSKCNL 850
SSVLKFLSVM KSSISGIDED VDNCDTSRMV ETRRANFAEE FAAAMHLSYP 900
ELMQTVAGEA KREAEERHEK KKMRAALPLL DVGSNGFAFS F*
MBP-JmJ2 (Jumonji Fusion Protein 2)
(SEQ ID NO: 7)
MKIEEGKLVI WINGDKGYNG LAEVGKKFEK DTGIKVTVEH PDKLEEKFPQ 50
VAATGDGPDI IFWAHDRFGG YAQSGLLAEI TPDKAFQDKL YPFTWDAVRY 100
NGKLIAYPIA VEALSLIYNK DLLPNPPKTW EEIPALDKEL KAKGKSALMF 150
NLQEPYFTWP LIAADGGYAF KYENGKYDIK DVGVDNAGAK AGLTFLVDLI 200
KNKHMNADTD YSIAEAAFNK GETAMTINGP WAWSNIDTSK VNYGVTVLPT 250
FKGQPSKPFV GVLSAGINAA SPNKELAKEF LENYLLTDEG LEAVNKDKPL 300
GAVALKSYEE ELAKDPRIAA TMENAQKGEI MPNIPQMSAF WYAVRTAVIN 350
AASGRQTVDE ALKDAQTNSS SNNNNNNNNN NLGIEGRISE FMLELDGRTL 400
TFEQFRECCL KPNLPAIIRH AAVDSTTNSV GGSGPSPYFS PLGDMQSHLS 450
PIGVVNLFGG DHVVPATESP ATVASDSLKD GISEGHMKCS KLRLFEVIER 500
WRGSPTLVYV KDWHMQSDLE AVSSCVGAPD LRGVSSVVAS DAEEGNAAVC 550
NGRRAVVHGG NLYCVPCYLG PDWMDEFCRF SQHGDSKYRY FGEEESDYRF 600
AYIGPPRSWT PLHFDVFGTY SWSLNVCGEK LWFFPTPEGN QTLLRGGLHG 650
VALAPDIRTT AGAELWTVTQ YPGDLVFVPS CYLHQVHNVK GSCFTLPQTR 700
ETANVAATSC EGIESVSTPN ESVVDLVISI NHNWCNEWCV ERMVDAFCRD 750
ANRLWMLLGD EVRLTLFGDD VGAWHDHVEN LLMGGTNWNF GCIRSFLVYR 800
LQVLRSSGPT TAEGDNVRRL IELCLGKVNE TEMRVAHCVR G*

The active TbJmJ1 protein (having methylarginine demethylation activity) is present in Jumonji Fusion Protein 1 (SEQ ID NO:6) from amino acid position 392 (indicated by underlined M) to the last amino acid at position 941 (F). The active TbJmJ1 protein sequence is provided as SEQ ID NO:8.

The active TbJmJ2 protein (having methylarginine demethylation activity) is present in Jumonji Fusion Protein 2 (SEQ ID NO:7) from amino acid position 392 (indicated by underlined M) to the last amino acid at position 841 (G). The active TbJmJ2 protein sequence is provided as SEQ ID NO:9.

Jumonji Fusion Protein 3 is Jumonji Fusion Protein 1 in which histidine at position 745 is changed to alanine. Jumonji Fusion Protein 4 is Jumonji Fusion Protein 2 in which histidine at position 613 is changed to alanine. The amino acid sequences of proteins Jumonji fusion protein 3 and 4, are provided as SEQ ID NO:10 and SEQ ID NO:11 respectively. These proteins (Jumonji fusion proteins 3 and 4) do not exhibit methylarginine demethylation activity and can therefore be used as negative controls for the presence of this enzymatic activity. The amino acid sequence of inactive TbJmJ1 enzyme in which histidine at position354 (of SEQ ID NO:8) is changed to alanine is provided in SEQ ID NO:12. The amino acid sequence of inactive TbJmJ1 enzyme in which histidine at position 222 (of SEQ ID NO:9) is changed to alanine is provided in SEQ ID NO:13.

The fusion proteins disclosed are unique recombinant, genetically engineered products capable of removing methyl groups from arginine residues in proteins and peptides. The catalytic activity, specifically with regard to methylarginine, is presently unavailable in any other form. The TbJmJ enzymes described above will be essential for the production of first-in-class chemical compounds capable of interfering with naturally occurring enzymes in plants and animals including humans. The use of TbJmJ enzymes as model target molecules will facilitate the design of compounds capable of arginine demethylation inhibition.

In one embodiment, the present disclosure provides a method for determining if a test sample contains demethylation activity comprising a) contacting the test sample with a peptide that is suitable to act as a substrate for a methylarginine demethylase; and b) using an antibody that is specific for either methylated arginine in a GRG motif or is specific for a non-methylated arginine in a GRG motif, determining if there is a change in the methylation level of the substrate peptide, and if a change (increase in binding of anti-RG or a decrease in binding of anti-mRG, or both) is observed, then identifying the test sample as containing demethylation activity.

In one embodiment, this disclosure provides a method for determining if a test sample contains demethylation activity comprising: a) contacting the test sample with a peptide that is suitable to act as a substrate for a methylarginine demethylase; and b) using an antibody that is specific for methylated arginine in a GRG motif and an antibody that is specific for a non-methylated arginine in a GRG motif, and based on the differential binding of the two antibodies, determining if methylarginine demethylation activity is present in the test sample.

In one embodiment, this disclosure provides a method for determining if a test sample contains demethylation activity comprising: a) contacting the test sample with a peptide substrate for a methylarginine demethylase; b) separately contacting a positive control exhibiting demethylation activity with the peptide substrate for a methylarginine demethylase; c) optionally, separately contacting the peptide substrate with a negative control for demethylation activity; d) using an antibody that is specific for either methylated arginine in a GRG motif (anti-mRG) or is specific for a non-methylated arginine in a GRG motif (anti-RG), determining if there is a change in the methylation level of the substrate peptide. If a change is observed in the binding of the anti-RG or the anti-mRG antibody (increase in binding of anti-RG or decrease in binding of anti-mRG, or both) in the test sample as a result of incubation of the substrate with the test sample, then that identifies the presence of demethylation activity in the test sample. A negative control (such as a protein lacking methylarginine demethylation activity, a test sample not known to have demethylation activity, or a reaction mixture without the test sample) may be run in parallel to confirm the reaction products are not due to non-specific reactions. If no change is observed in the binding of the anti-RG or the anti-mRG antibody in the test sample as a result of incubation of the substrate with the test sample, then a positive control (such as a Jumonji protein as described herein) may be used in parallel to confirm the absence of demethylase activity seen in the test sample (i.e., negative result) is credible. In one embodiment, the positive control is a protein comprising or consisting of the sequence of SEQ ID NO:8 or SEQ ID NO:9, SEQ ID NO:6 or SEQ ID NO:7. In one embodiment, the negative control is a protein comprising or consisting of the sequence of SEQ ID NO:12 or SEQ ID NO:13, SEQ ID NO:10 or SEQ ID NO:11.

The co-development of an arginine demethylation assay disclosed herein incorporates one or more of the fusion proteins described above as comparative standards for the relative quantitative assessment of naturally occurring arginine demethylating activity in biological cells, tissues and fluids.

### EXAMPLE 2

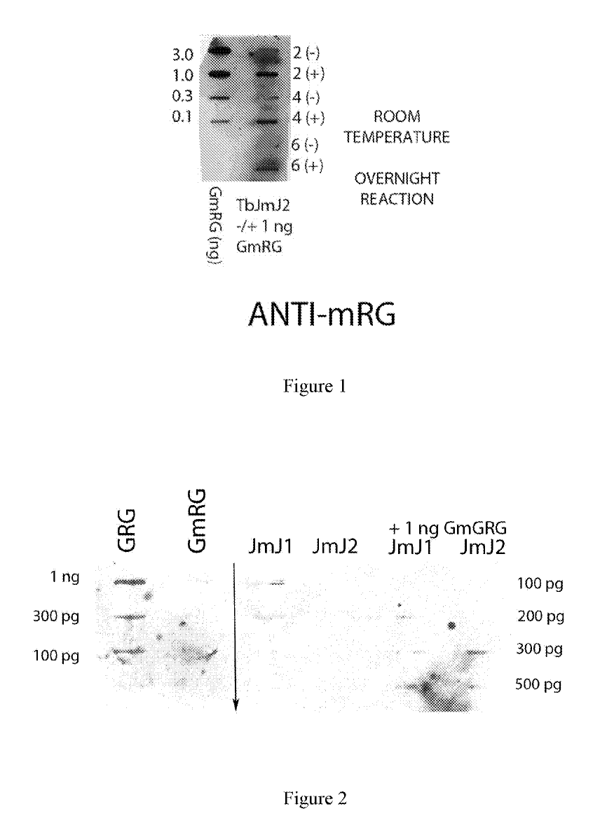

This example illustrates the method of this disclosure. Description: In the upper illustration (FIG. 1), methylarginine-specific antibody has been used to probe reaction mixtures containing 2, 4 and 6 μl of purified TbJmJ2±1ng of methylarginine peptide (GmRG). In the left column, standard amounts of the GmRG peptide (0.1 to 3 ng) have been applied to PVDF membrane. In the column to the right, reaction mixtures of TbJmJ2 incubated for 20 hours (22.5° C.) with (+) 1 ng GmRG or without (−) added GmRG were applied to the PVDF membrane. Antibody binding was determined by standard chemi-luminescence assay with light reaction captured using X-ray film detection. Comparing the (+1 ng) lanes on the right with the 1 ng standard on the left indicates a concentration-dependent decrease in the methylation status of the GmRG peptide. In the lower illustration (FIG. 2), a similar experiment was analyzed using anti-RG, antibody specific for the non-methylated form of the glycine- and arginine-rich peptide (GRG). Anti-RG used under these conditions, binds only to non-methylated GRG peptide (cf. GRG and GmRG in 2 columns on the left). The results from the incubation mixtures in the four columns on the right side of the illustration demonstrate (1) that the anti-RG does not bind to TbJmJ1 or TbJmJ2 (100 to 500 pg) and (2) that both TbJmJ enzymes are capable of converting a portion of GmRG to GRG, giving a positive reaction with anti-RG in a dose-dependent manner.

In an experiment, the methylarginine peptide was incubated in reaction tubes containing homogenates of cultured cells and the experiment was carried out as described above. A time-dependent reduction in the binding of the methylarginine-specific antibody to the reaction tube contents at the end of a 60-minute incubation period (30°) was observed, similar to that observed in FIG. 1 using purified TbJmJ2. The quantity and mass of the methylarginine peptide was confirmed, ruling out any proteolysis of the peptide.

While the present disclosure has been described through examples, routine modifications to the disclosure are within the purview of those skilled in the art and such modifications are intended to be within the scope of this disclosure.

1. A method for determining if a test sample contains methylarginine demethylation activity comprising:
a) contacting the test sample with a peptide substrate for methylarginine demethylase activity, said substrate comprising a GRG motif, wherein the R is methylated;
b) separately contacting the peptide substrate with a positive enzyme control for methylarginine demethylase activity; and
c) using an antibody that is specific for methylated arginine in a GRG motif or specific for non-methylated arginine in a GRG motif, determining if there is a decrease in the methylation level of the substrate peptide by the test and the positive enzyme control,
wherein a decrease in the methylation status of the peptide substrate by the test sample identifies the presence of methylarginine demethylation activity in the test sample, and a lack of decrease in the methylation status of the peptide substrate combined with a decrease of methylation status by the positive enzyme control identifies the absence of methylarginine demethylation activity in the test sample.

2. A method for determining if a test sample contains methylarginine demethylation activity comprising:
a) contacting the test sample with a peptide substrate for a methylarginine demethylase activity;
b) separately contacting the peptide substrate with a positive enzyme control for methylarginine demethylase activity;
c) separately contacting the peptide substrate with a negative control for methylarginine demethylase activity; and
d) using an antibody that is specific for methylated arginine in a GRG motif or specific for a non-methylated arginine in a GRG motif, determining if there is a decrease in the methylation level of the substrate peptide for the test sample, the positive enzyme control, and the negative control,
wherein a decrease in the methylation status of the substrate by the test sample combined with a lack of decrease in the methylation status of the substrate by the negative control identifies the presence of methylarginine demethylation activity in the test sample, and a lack of decrease in the methylation status of the substrate combined with a decrease in the methylation status by the positive enzyme control identifies the absence of methylarginine demethylation activity in the test sample.

3. The method of claim 1, wherein the positive enzyme control for methylarginine demethylase activity is a protein comprising the sequence of SEQ ID NO:8 or SEQ ID NO:9.

4. The method of claim 1, wherein the positive enzyme control for methylarginine demethylase activity is a protein comprising the sequence of SEQ ID NO:6 or SEQ ID NO:7

5. The method of claim 4, wherein the substrate peptide has from 5 to 30 amino acids comprising 2 to 14 GRG repeats.

6. The method of claim 5, wherein the substrate peptide comprises the sequence of SEQ ID NO:1, or the sequence of SEQ ID NO:14.

7. The method of claim 2, wherein the negative control for methylarginine demethylase activity is a protein comprising the sequence of SEQ ID NO: 12 or SEQ ID NO:13.

8. The method of claim 2, wherein the negative control for methylarginine demethylase activity is a protein comprising the sequence of SEQ ID NO: 10 or SEQ ID NO:11.

9. A method for identifying agents that affect methylarginine demethylase activity comprising:
a) incubating the following:
i) a methylarginine demethylase, wherein the methylarginine demethylase is a protein comprising the sequence of SEQ ID NO: 8 or SEQ ID NO:9;
ii) a peptide or protein substrate comprising a methylarginine in a GRG motif that can be demethylated by the demethylase;
iii) one or more test agents
b) using an antibody that is specific for methylated arginine in a GRG motif or specific for a non-methylated arginine in a GRG motif, determining if the presence of the test agent inhibits or enhances the demethylase activity.

10. The method of claim 9, wherein the methylarginine demethylase is a protein comprising the sequence of SEQ ID NO:6 or SEQ ID NO:7.

11. The method of claim 9, wherein the peptide substrate has from 5 to 30 amino acids comprising 2 to 14 GRG repeats.

12. The method of claim 11, wherein the substrate peptide comprises the sequence of SEQ ID NO:1 or SEQ ID NO:14.

13. A kit for aiding in determining the arginine methylation status of a test sample comprising:
a) a peptide substrate for an methylarginine demethylase;
b) an enzyme having methylarginine demethylase activity, wherein the methylarginine demethylase is a protein comprising the sequence of SEQ ID NO: 8 or SEQ ID NO:9;
c) an antibody that i) specifically recognizes a GRG motif in a peptide or protein in which the arginine is not methylated but does not recognize the GRG motif in a peptide or protein in which the arginine is methylated; and/or ii) specifically recognizes a GRG motif in a peptide or protein in which the arginine is methylated but does not recognize the GRG motif in a peptide or protein in which the arginine is not methylated;
d) optionally one or more buffers;
e) optionally instructions for carrying out the test.

14. The kit of claim 13, wherein the peptide substrate has from 5 to 30 amino acids comprising 2 to 14 GRG repeats.

15. The kit of claim 14, wherein the substrate peptide comprises the sequence of SEQ ID NO:1 or SEQ ID NO:14.

16. The kit of claim 13, wherein the enzyme having methylarginine demethylase activity is a protein comprising the sequence of SEQ ID NO:6 or SEQ ID NO: 7.

17. The kit of claim 13, further comprising a negative control for methylarginine demethylase activity, wherein the negative control is a protein comprising the sequence of SEQ ID NO:12 or SEQ ID NO:13.

18. The kit of claim 13, wherein the negative control is a protein comprising the sequence of SEQ ID NO:10 or SEQ ID NO: 11.

19. The method of claim 2, wherein the positive enzyme control for methylarginine demethylase activity is a protein comprising the sequence of SEQ ID NO:8 or SEQ ID NO:9.

20. The method of claim 2, wherein the positive enzyme control for methylarginine demethylase activity is a protein comprising the sequence of SEQ ID NO:6 or SEQ ID NO:7.
